(19) United States
(12) Patent Application Publication
(10) Pub. No.: US 2023/0405103 A1
(43) Pub. Date: Dec. 21, 2023

(54) COXIELLA BURNETII AVIRULENT NINE MILE PHASE II VIABLE BACTERIA AND METHODS OF USE

(71) Applicant: BOARD OF REGENTS, THE UNIVERSITY OF TEXAS SYSTEM, Austin, TX (US)

(72) Inventor: Guoquan F. Zhang, San Antonio, TX (US)

(21) Appl. No.: 17/969,740

(22) Filed: Oct. 20, 2022

Related U.S. Application Data
(60) Provisional application No. 63/270,532, filed on Oct. 21, 2021.

(51) Int. Cl. A61K 39/02

(52) U.S. Cl. CPC A61K 39/0208; A61K 2039/543

(57) ABSTRACT

Certain embodiments are directed to the use of avirulent LPS phase II viable bacteria as a live attenuated vaccine against human Q fever and method of immunizing a subject with said vaccine.

### PRIORITY PARAGRAPH

This application claims priority to U.S. Provisional Patent Application Ser. No. 63/270,532 filed Oct. 21, 2021, which is incorporated herein by reference in its entirety.

### STATEMENT REGARDING FEDERALLY FUNDED RESEARCH

This invention was made with government support under R01AI134681, R21AI130347, and R21AI137504 awarded by the National Institutes of Health (NIH)/National Institute of Allergy and Infectious Diseases (NIAID). The government has certain rights in the invention.

### BACKGROUND

Embodiments of the invention are related to the field medicine, immunology, and veterinary medicine, and in particular to vaccines for Q fever.

Coxiella burnetii is an obligate intracellular bacterium that causes flu-like zoonosis, Q-fever. Cattle, sheep, and goat are the primary reservoirs for human infections. The enhanced stability of the organism in aerosol contribute to their potential to initiate a pandemic and therefore classified as a Tier 2 select agent by the United States Centers for Disease Control and Prevention (CDC) [1]. Previous reports suggest that intertropical areas are commonly prone to C. burnetii infection [2] [3]. Q fever outbreak in Netherlands from 2007 to 2010 resulted in 4,107 notifications indicating that Q fever remains a significant threat for public health [4] [5]. Mostly, serological assays are used to diagnose acute infection and most of these patients recover after treatment with 100 mg dose of doxycycline twice a day for two weeks [6]. In case of chronic Q fever, 100 mg dose of doxycycline twice daily and 200 mg hydroxychloroquine three times a day for a minimum of 18 months and longer in immune-compromised patients [7]. To prevent these complications, a wide-range preventive vaccine is critical, specifically for those at risk due to occupation, such as veterinarians, meat processing plant workers, sheep and dairy workers, livestock farmers, and researchers at facilities housing sheep.

Q-Vax® is the only licensed vaccine for human vaccination, which is a formalin-inactivated whole cell vaccine, produced from the Henzerling Phase I strain and can provide reliable protection against Q-fever [8]. However, the vaccine induces adverse reactions such as subcutis swelling, erythema and induration in subjects pre-immune to C. burnetii [9]. Therefore, it is required to test for sensitization to Q-fever antigens using Q-VAX® Skin Test in every individual prior to immunization. In terms of veterinary vaccines, Chlamyvax FQ® and C oxevac®, are commercially available. Chlamyvax FQ is prepared as an oil emulsion with Chlamydophila abortus and inactivated phase II C. burnetii and is approved in France [10]. However, Chlamyvax FQ did not show effectiveness in protection against abortion and C. burnetii shedding in milk, feces, placenta, and vaginal secretions compared to the unvaccinated control group [11]. The inventor has developed a Phase-I LPS-targeted peptide mimic vaccine based on the concept of reverse vaccinology. Although vaccination with the mimotope vaccine elicited significant protection against C. burnetii infection in mice, the levels of protection are comparatively lower than the formalin-inactivated NMI whole cell vaccine (PIV) [12], suggesting that other antigenic components beyond LPS in PIV might be necessary for coffering complete protection.

There remains a need for improved vaccines against Q-fever.

### SUMMARY

In the study described in the Example section below, the Inventor examined if viable avirulent NMII bacteria can elicit protective immunity against virulent C. burnetii infection in mouse models of Q fever. Interestingly, viable NMII bacteria elicited a similar level of long-term protection against various virulent C. burnetii infections as the PIV in mice. In addition, viable NMII bacteria-induced protective immunity depends on both B cells and T cells but T cells may play a critical role in controlling bacterial replication. Certain embodiments are directed to the use of avirulent NMII viable bacteria as a live attenuated vaccine against human Q fever.

Certain embodiments are directed to a Q fever vaccine comprising a live avirulent Coxiella burnetii (C. burnetii) strain having a phase II LPS phenotype. The avirulent C. burnetii strain can be a nine mile phase II strain (NIVIII).

Other embodiments are directed to methods of immunizing a mammal against q fever comprising administering a live avirulent Coxiella burnetii (C. burnetii) strain having a phase II LPS phenotype to a subject. In certain aspects the avirulent C. burnetii strain can be a nine mile phase II strain (NMII). In a particular aspect the vaccine is administered is by intranasal administration.

As used herein the terms “treat” and “prevent”, and the like, refer to any and all uses which remedy a condition or symptoms, prevent the establishment of a condition or disease, or otherwise prevent, hinder, retard, or reverse the progression of a condition or disease or one or more undesirable symptoms thereof in any way whatsoever. Thus the terms “treat” and “prevent” and the like are to be considered in their broadest context. For example, treatment does not necessarily imply that a subject is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms. In the context of the present invention, symptoms that may be ameliorated, reversed, prevented, retarded or the linked include but are not limited to fever.

The term “subject” as used herein refers to a mammal, more particularly a human, who can benefit from a vaccine or method disclosed herein. The term “subject” as used herein also includes non-human primates, livestock animals (e.g. cattle, dairy cows, horses, sheep, pigs), laboratory test animals (e.g. mice, rabbits, rats, guinea pigs), companion animals (e.g. dogs, cats), wild animals and captive wild animals. A subject regardless of whether a human or non-human mammal may be referred to herein as an individual, subject, animal, patient, or recipient.

As used herein the term “immunoprotective” includes within its meaning a non-toxic but sufficient amount or dose of a composition or vaccine to elicit or induce a protective immune response in a subject. The exact amount or dose required will vary from subject to subject depending on factors such as the species being treated, the age and general condition of the subject, the particular vaccine being administered and the mode of administration and so forth. For any given case, an appropriate immunoprotective amount or dose may be determined by one of ordinary skill in the art using only routine experimentation.

The term “vaccine” as used herein, is a broad term and is used in its ordinary sense, including, without limitation, avirulent C. burnetii having a phase II LPS phenotype or conjugates of the same comprising avirulent C. burnetii linked to an immunogenic carrier, optionally formulated with adjuvants, diluents, excipients, carriers, and other pharmaceutically acceptable substances. The term “pharmaceutically acceptable” is used to refer to a non-toxic material that is compatible with a biological system such as a cell, cell culture, tissue, or organism.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”′.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1C. Viable avirulent NMII bacteria interfere virulent C. burnetii NMI infection. (A) BALB/c mice were IP infected with (1) 1×10⁷ virulent NMI, (2) 1×10⁷ avirulent NMII, (3) 1×10⁷ NMII 3 days before infection with 1×10⁷ NIVII, (4) 1×10⁷ NMI+1×10⁷ NMII, or (5) 1×10⁷ NMI 3 days before infection with 1×10⁷ NMII. Splenomegaly (B) and bacterial burden in the spleen (C) were evaluated at 14 days post infection (dpi). Splenomegaly is expressed as percent of (spleen weight/body weight). Bacterial burden was determined by real-time quantitative PCR (qPCR) and is expressed as log₁₀ C. burnetii com1 genomic copy numbers. Each experimental group includes four mice, with error bars representing the standard deviations from the means. *, P<0.05; **, P<0.01; ***, P<0.001; ****, P<0.0001, as determined by welch-t test using Graph pad.

FIG. 2A-2E. Mice immunization with viable NMII bacteria conferred significant protection against virulent NMI challenge. (A) BALB/c mice were IP vaccinated with 1×10⁷ GE of viable NMII and IP challenged with 1×10⁷ GE of NMI at 7, 14, and 28 days post vaccination (dpv), respectively. Body weight ratio (B), splenomegaly ((C) % of spleen weight/body weight) and bacterial burden in the spleen ((D) log₁₀ C. burnetii com1 gene copy numbers, (E) NMI-LPS-specific CBU 0680 gene copy numbers) were evaluated at 14 dpi. Each experimental group includes four mice, with error bars representing the standard deviations from the means*, P<**, P<0.01; ***, P<0.001; ****, P<0.0001, as determined by welch-t test using Graph pad.

FIG. 3A-3E. Mice immunized with lower doses of viable NMII bacteria also conferred significant protection against virulent C. burnetii challenge. (A) BALB/c mice were IP vaccinated with 10¹, 10³, 10⁵ or 10⁷ GE of NMII and IP challenged with 1×10⁷ GE of NMI at 28 dpv. Body weight ratio (B), splenomegaly ((C), % of spleen weight/body weight) and bacterial burden in the spleen ((D) log₁₀ C. burnetii com1 gene copy numbers, (E) NMI-LPS-specific CBU 0680 gene copy numbers) were evaluated at 14 dpi. Each experimental group includes four mice, with error bars representing the standard deviations from the means. *, P<0.05; **, P<***, P<0.001; ****, P<0.0001, as determined by welch-t test using Graph pad.

FIG. 4A-4F. Viable NMII bacteria induce antibody response in mice against NMI antigens. The concentration of C. burnetii WI-specific IgM (B), IgG (C), IgG1 (D), IgG2a (E), and IgG3 (F) in serum samples from (A) different dose of viable NMII-vaccinated mice and challenged with NMI at 14 dpi were analyzed by ELISA. Antibody concentration is expressed as μg/mL. Statistical analysis was performed between NMII immunized and unimmunized group. *, P<0.05; **, P<0.01; ***, P<0.001; ****, P<0.0001, as determined by welch-t test using Graph pad.

FIG. 5A-5D. Mice immunization with viable NMII via different routes conferred similar protection against virulent NMI challenge. (A) BALB/c mice were vaccinated with 10⁵ GE of viable NMII via IP, IN, IM or SQ routes and IP challenged with 1×10⁷ GE of NMI at 28 dpv. Body weight ratio (B), splenomegaly ((C) % of spleen weight/body weight) and bacterial burden (log₁₀ C. burnetii com1 gene copy numbers) in the spleen (D) were evaluated at 14 dpi. Each experimental group includes four mice, with error bars representing the standard deviations from the means. *, P<0.05; **, P<0.01; ***, P<0.001; ****, P<0.0001, as determined by welch-t test using Graph pad.

FIG. 6A-6D. Mice immunization with viable NMII bacteria provides similar protection as formalin-killed PIV. (A) BALB/c mice were IP vaccinated with 1×10⁵ GE of viable NMII, SQ immunized with 10 μg of PIV or SQ immunized with 10 μg of PIIV and IP challenged with 1×10⁷ GE of NMI at 14 dpv. Body weight ratio (B), splenomegaly (C) % of spleen weight/body weight) and bacterial burden (log₁₀ C. burnetii com1 gene copy numbers) in the spleen (D) were evaluated at 14 dpi. Each experimental group includes four mice, with error bars representing the standard deviations from the means. *, P<0.05; **, P<0.01; ***, P<0.001; ****, P<0.0001, as determined by welch-t test using Graph pad.

FIG. 7A-7D. Mice immunization with viable NMII bacteria conferred long-term protection against virulent NMI challenge. (A) BALB/c mice were SQ vaccinated with 10⁵ GE of viable NMII, 10 μg of PIV, PBS or adjuvant along and IP challenged with 10⁷ GE NMI at 120 dpv. Body weight ratio (B), splenomegaly (C) % of spleen weight/body weight) and bacterial burden (log₁₀ C. burnetii com1 gene copy numbers) in the spleen (D) were evaluated at 14 dpi. Each experimental group includes four mice, with error bars representing the standard deviations from the means. *, P<0.05; **, P<0.01; ***, P<0.001; ****, P<0.0001, as determined by welch-t test using Graph pad.

FIG. 8A-8D. Mice immunized with viable NMII bacteria confers protection against other virulent strains of C. burnetii. (A) BALB/c mice were IP vaccinated with 1×10⁵ GE of viable NMII and IP challenged with 1×10⁷ GE of NMI, Scurry or Priscilla at 28 days dpv. Body weight ratio (B), splenomegaly (C) % of spleen weight/body weight) and bacterial burden (log₁₀ C. burnetii com1 gene copy numbers) in the spleen (D) were evaluated at 14 dpi. Each experimental group includes four mice, with error bars representing the standard deviations from the means. *, P<0.05; **, P<0.01; ***, P<0.001; ****, P<0.0001, as determined by welch-t test using Graph pad.

FIG. 9A-9D. Adoptive transfer of serum and splenocytes from NMII immunized mice induce protection against NMI challenge in naïve mice. (A) BALB/c mice were IP transferred with sera or splenocytes from PBS or viable NMII-immunized mice and IP challenged with 1×10⁷ GE of NMI at 3 days post adoptive transfer. Body weight ratio (B), splenomegaly ((C) % of spleen weight/body weight) and bacterial burden (log₁₀ C. burnetii com1 gene copy numbers) in the spleen (D) were evaluated at 14 dpi. Each experimental group includes four mice, with error bars representing the standard deviations from the means. *, P<0.05; **, P<0.01; ***, P<0.001; ****, P<0.0001, as determined by welch-t test using Graph pad.

FIG. 10A-10D. Viable NMII bacteria induced partial protection in B cell and T cell knockout mice against virulent NMI challenge. (A) C57BL/6J wild type (WT) and B cell, T cell, CD4⁺ T cell or CD8⁺ T cell deficient mice were IP immunized with 10⁵ GE of viable NMII and IP challenged with 1×10⁷ GE of NMI at 28 dpv. Additionally, PBS-immunized WT mice were IP challenged with 1×10⁷ GE of NMI served as unimmunized controls. Body weight ratio (B), splenomegaly ((C) % of spleen weight/body weight) and bacterial burden (log₁₀ C. burnetii coral gene copy numbers) in the spleen (D) were evaluated at 14 dpi. Each experimental group includes four mice, with error bars representing the standard deviations from the means. *, P<0.05; **, P<0.01; ***, P<0.001; ****, P<0.0001, as determined by welch-t test using Graph pad.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to intimate that the scope of the disclosure, including the claims, is limited to that embodiment.

Among the limited virulence factors reported in C. burnetii, LPS is the major virulence factor where the O-antigen plays a major role in pathogenesis [13]. Moreover, phase I LPS was able to confer protective immunity against virulent C. burnetii infection [14]. Virulent C. burnetii NMI strain undergo phase variation after serial passage in embryonated eggs, which a non-reversible shift from full-length LPS of virulent NMI bacteria to avirulent NMII with truncated LPS lacking O-antigen and several core sugars [15]. Whole-genome sequencing revealed that NMII has a ˜26-kb chromosomal deletion, compared to NMI, where the deleted region eliminates several LPS biosynthetic genes resulting in the production of truncated LPS [16-20], therefore NMII bacteria cannot revert to phase I LPS-expressing C. burnetii. Because of clonality, avirulence in a guinea pig model of infection and lack of phase reversion, NMII strain is considered as a biosafety level-2 (BSL-2) pathogen [16,21]. Vishwanath and Hackstadt demonstrated that NMI with smooth LPS was resistant to complement-mediated killing by human serum, whereas NMII with rough LPS was killed by serum complement. The inventor's earlier studies indicated that the rate of replication of NMII in mouse immune cells including neutrophils and B cells is different with NMI. Infection in BALB/c mice indicated that 7 of NMI bacteria can induce high splenomegaly while 10⁷ of NMII bacteria do not induce splenomegaly or fever in guinea pigs [25]. Moos and Hackstadt reported that NMII did not elicit fever or seroconversion except with very large inocula (10⁸), and viable organisms could not be recovered at 30 days post infection in guinea pig model. Masako et al have been shown that splenomegaly and bacterial burden in the spleen were undetectable in 1×10⁵ of NMII bacteria infected Wt, B cell deficient, NK cell deficient, TNFα deficient and IFNγ deficient mice at 28 days post-infection. Although 1×10⁵ of NMII bacteria can induce splenomegaly and bacterial burden in T cell deficient mice, the splenomegaly and bacterial numbers were significantly lower than virulent NMI-infected mice, indicating that the avirulent NMII strain will be cleared at 28 day post-infection. Additionally, only very high dose (10⁸) of NMII can induce splenomegaly in SCID mice, which lacks complete adaptive immunity [28]. These data indicate that NMII bacteria possess the potential to be a safe and effective live attenuated vaccine against virulent C. burnetii infection [25,29]. In general, live attenuated vaccines are highly efficient as they mimic the natural infection in host without causing pathogenicity [30]. Many successful live bacterial vaccines have been approved by FDA and are used extensively for decades to prevent various respiratory (BCG) and enteric (Cholera) pathogenic diseases [31]. Considering the advantages of live attenuated vaccines, no-pathogenic NMII bacteria might be useful for development of a live attenuated vaccine against Q fever.

This study described below explored if viable C. burnetii avirulent Nine Mile phase II (NMII) can elicit protective immunity against virulent NM phase I (NMI) infection. Interestingly, mice immunized with viable NMII elicited significant protection against NMI infection at different time points post-immunization. Viable NMII induced a dose-dependent NMI-specific IgG response in mice but all doses of NMII-immunized mice conferred a similar level of protection. Comparing different route of immunization indicated that intranasal immunized mice showed significant higher levels of protection than other immunization route. The observation that viable NMII induced a similar level of long-term protection against NMI challenge as the formalin-inactivated NMI vaccine (PIV) suggests that viable NMII bacteria can induce a similar level of long-term protection against virulent NMI challenge as the PIV. Viable NMII also induced significant protection against challenge with virulent Priscilla and Scurry strains, suggesting that viable NMII can elicit broad protection. Immune sera and splenocytes from viable NMII-immunized mice are protective against NMI infection but immune sera receiving mice did not control NMI replication. Additionally, viable NMII conferred a comparable level of protection in Wild type, CD4⁺ T cell deficient, and CD8⁺ T cell deficient mice, and partial protection in B cell deficient mice. However, NMII-immunized T cell deficient mice were unable to prevent C. burnetii replication. Thus, both B cells and T cells are required for viable NMII-induced protective immunity but T cells may play a critical role. Collectively, this study demonstrates the feasibility of using avirulent NMII as a live attenuated vaccine against human Q fever.

Utilizing a live attenuated vaccine that expresses the similar antigenic determinants as the virulent C. burnetii but lacks the virulence factors, which reduces the risk of infection while can provide maximum protection might be an ideal paradigm for safe vaccination against human Q fever. Since C. burnetii NMII strain derived from NMI, which exhibit truncated LPS, fulfills the criteria required for a live attenuated vaccine candidate. Because of the genomic deletion of LPS biosynthetic genes, NMII bacteria produce truncated LPS, lacking the major virulence factor [15]. NMII infection in a guinea pig model did not induce splenomegaly or fever response, indicating that NMII is a non-pathogenic strain [25]. The inventor's previously study also demonstrated that the infection rate of neutrophils by NMII is similar to that of NMI, yet the intracellular bacterial load of NMII is significantly less than NMI, showing that NMII is more readily cleared from neutrophils than NMI [37]. Additionally, proteomic analysis has indicated that NMII and NMI respond differentially to stress in L929 cells, indicating that the intracellular survival of both strains are distinct [38]. Regarding protective immunity, NMII infection induces toll-like receptor 4 independent dendritic cell maturation with high IL-12 and TNF production [29], implying that NMII might activate dendritic cell mediated T-cell response in host. These findings suggest that viable NMII bacteria may be able to activate the immune system against subsequent infections with virulent C. burnetii isolates without cause clinical illness. The study described below aimed to investigate the feasibility of using viable NMII bacteria as a live attenuated vaccine against virulent C. burnetii infection in a mouse model.

It is notable that mice immunized with viable NMII bacteria at 3 days prior to NMI challenge significantly reduced splenomegaly but were unable to protect against bacterial replication. These results may be due to C. burnetii is an intracellular pathogen, which can establish a vacuolar biogenesis after invading host cells for its intracellular replicating, resulting the bacteria escaped from the host immune system at early stage of infection [39,40]. However, mice immunized with viable NMII bacteria provided significant protection against NMI-infection induced splenomegaly and bacterial replication from 7 days up to 120 days post vaccination with the trend showing that protection increases over time. These results suggest that viable NMII bacteria were able to confer significant protection against NMI challenge and protection can lasting at least for 4 months tested. It is important to note that immunization using different doses of NMII indicated a similar level of protection regardless the immunization dose and that even a very low infection dose of 10¹ GE was sufficient to induce protection. Collectively, these findings demonstrate the possibility of using avirulent NMII stain as a live attenuated vaccine against human Q fever.

The major problem with the existing vaccine, Q-VAX®, is that subcutaneous or intra-dermal injection of the vaccine induces strong inflammatory response at the site of inoculation, especially in individuals who have been exposed to C. burnetii prior vaccination [9]. An adverse events following immunization (AEFI) study conducted in QVAX® vaccinated veterinary, and animal science students, at Australian universities showed that QVAX® induced severe local and systemic AEFI [41]. These data provide strong evidence to support that Q-VAX® has the potential to induce AEFI in humans. Similar to those observations from human vaccinees, we have also observed nodule-like inflammation reactions at the sites of PIV injected mice, however the adverse reactions were not observed in the site of SQ immunized mice with viable NMII bacteria (data not shown). These observations suggest that a viable NMII bacteria-based vaccine is potentially safer than PIV for immunization of humans. However, further studies needed to examine whether viable NMII bacteria can induce local and systemic adverse reactions in C. burnetii sensitized animals.

Generally, mucosal vaccines can induce both systemic and mucosal immunity, including antigen specific IgA response, especially at mucosal surfaces, which are the frontlines of host defense against multiple pathogens [42]. Additionally, the intranasal vaccination method is a less invasive route for immunization of humans and has been widely approved vaccination route for human vaccines. Interestingly, it was found that IN immunization with viable NMII induced a stronger protective immunity against NMI infection than IP, IM and SQ immunization with NMII. A previous study demonstrated that even high-doses of NMII infection (aerosol infection with 1×10⁹ of bacteria) did not induce significant inflammatory response and cause clinical disease in immunodeficient SCID mice [37]. These data suggest that IN immunization is safe and effective for human vaccination with the NMII live attenuated vaccine. To further validate the efficacy of viable NMII vaccine by IN immunization, the inventor examined if mice IN vaccinated with viable NMII bacteria would provide similar levels of broad protection as the PIV against virulent C. burnetti Priscilla and Scurry strain challenge. The results indicate that IN immunization with viable NMII bacteria conferred a similar level of significant protection as the PIV against challenge with both virulent Priscilla and Scurry strains. Since both Priscilla and Scurry strains are considered as chronic Q fever associated isolates, the observation that viable NMII bacteria conferred significant protection against both Priscilla and Scurry strains provided further evidence to support that viable NMII bacteria can confer broad protection against both acute and chronic Q fever associated isolates. Additionally, this result further demonstrates that IN immunization would be a safe, effective and practicable vaccination method for using viable NMII bacteria as an attenuated live vaccine against human Q fever.

Vaccination is an active immune approach for simulation of the immune system to generate antigen-specific humoral and cellular immune responses, which can induce lasting-protective immunity against infectious diseases [43]. The major advantage of using a whole-cell vaccine is that it induces a long-term T cell response, in addition to antibody response [44]. Izzo et al. reported that Q-VAX® induces a long-lived T-cell response which is detectable for at least 8-10 years following vaccination. To understand the mechanism of the NMII live attenuated vaccine-induced protective immunity, we examined if transfer immune sera or splenocytes from viable NMII bacteria vaccinated mice would protect naive recipient mice against virulent NMI infection. The results indicated that both immune sera and splenocytes from NMII-immunized mice provided a similar level of protection against NMI-infection induced body weight-loss and splenomegaly. However, although splenocytes from NMII-immunized mice conferred significant protection against NMI-infection induced bacterial burden in the spleens, immune sera from NMII-immunized mice were unable to provide protection against the bacterial burden in naive recipient mice. These results suggest that both humoral and cellular immunities are involved in the viable NMII-induced protection but cellular immunity may play a critical role in controlling of bacterial replication in mice.

To identify which host immune components are crucial for viable NMII bacteria-induced protective immunity, we examined if B cell, T cell, CD4⁺ T cell or CD8⁺ T cell deficiency in mice will significantly affect the ability of viable NMII bacteria to confer protection against virulent NMI infection. The results demonstrated that viable NMII bacteria conferred a comparable level of protection against virulent NMI challenge in WT, CD4⁺ T cell deficient and CD8⁺ T cell deficient mice, and a partial protection in B cell deficient and T cell deficient mice. In addition, the result that the bacterial burden in the spleens from NMII-immunized T cell deficient mice was significantly higher than NMI-infected unimmunized WT mice suggests that T cells may play a critical role in controlling virulent C. burnetii replication in mice. Collectively, these results suggest that viable NMII bacteria-induced protective immunity depends on both B cells and T cells but T cells may play a critical role in controlling bacterial replication. Interestingly, our previous study also demonstrated that PIV conferred i) comparable levels of protection against NMI infection in WT, CD4⁺ T cell-deficient and CD8⁺ T cell-deficient mice, ii) partial protection against splenomegaly but no protection against bacterial replication in T cell-deficient mice and iii) no measurable protection in B cell-deficient mice. These data suggest that the mechanisms of viable NMII vaccine and PIV-induced protection may be similar and highlight the role of T cells for controlling bacterial replication in vaccine-induced protective immunity against this intracellular bacterial pathogen. In addition, our recent study demonstrated that MHC-II restricted PIV-specific CD4⁺ T cells and Th1 immune response play a crucial role in PIV-mediated protection against C. burnetii infection. These studies [14, 31, 44] suggest that anti-PI specific Abs play an important role in protection from the development of clinical disease at an early stage against C. burnetii infection, while the T cell-mediated Th1 immune response is critical for clearance and complete elimination of the organisms at the late stage of the infection. Therefore, novel vaccine approaches for Q fever should be focused on boosting both humoral and cellular immune responses.

In summary, the results demonstrated that viable NMII bacteria elicited a similar level of long-term protection against various virulent C. burnetii infections as the PIV in mice. In addition, viable NMII bacteria-induced protective immunity depends on both B cells and T cells but T cells may play a critical role in controlling bacterial replication. Thus, this study provided the first evidence to demonstrate the feasibility of using avirulent NMII viable bacteria as a live attenuated vaccine against human Q fever.

The vaccine may be administered by any suitable route such as, for example, intramuscularly, subcutaneously, orally or intranasally. In certain aspects the vaccine is administered intranasally. The immunoprotective amount of vaccine to be administered is typically determined on a case by case basis and may be determined by the skilled person without undue burden or the need for further invention. By way of example only, where the subject is a human, the immunoprotective amount may comprise between about 2 μg and about 200 μg or between about 5 μg and about 50 of an avirulent C. burnetii strain or a C. burnetii strain polysaccharide-carrier conjugate. For example, the immunoprotective amount may be about 2 μg, about 10 μg, about 20 μg, about 30 μg, about 40 μg, about 50 μg, about 60 μg, about about 80 μg, about 90 μg, about 100 μg, about 110 μg, about 120 μg, about 130 μg, about 140 μg, about 150 μg, about 160 μg, about 170 μg, about 180 μg, about 190 or about 200 μg of an avirulent C. burnetii strain or a C. burnetii strain polysaccharide-carrier conjugate. Immunogenicity of the vaccine can be assessed and monitored by a range of techniques available to those skilled in the art, including the synthesis of antibodies in the subject subsequent to administration of the vaccine.

The immunoprotective amount of the vaccine may be administered in a single dose or in a series of doses. Where more than one dose is required, the doses may be administered days, weeks or months apart, such as for example, 4 weeks apart. Thus, the vaccines can be administered as a single dose or in a series including one or more boosters. In embodiments in which the vaccine comprises an avirulent C. burnetii strain polysaccharide-carrier conjugate, typically only one or two doses of an immunoprotective amount of vaccine is required to achieve the desired protective or therapeutic effect.

The dosage of vaccine to be administered to a subject and the regime of administration can be determined in accordance with standard techniques well known to those of ordinary skill in the pharmaceutical and veterinary arts, taking into consideration such factors as the intended use, particular antigen, the adjuvant (if present), the age, sex, weight, species, general condition, prior illness and/or treatments, and the route of administration. Preliminary doses can be determined according to animal tests, and the scaling of dosages for human administration is performed according to art-accepted practices such as standard dosing trials. The dosage depends on the specific activity of the vaccine and can be readily determined by routine experimentation.

Vaccine compositions of the present disclosure are typically sterile and may contain one or more pharmaceutically-acceptable carriers, such as one or more compatible solid or liquid fillers, diluents or encapsulating substances which are suitable for administration to a subject. Formulation of vaccines of the present disclosure into pharmaceutical compositions can be accomplished using methods known in the art. The vaccine compositions can also contain one or more adjuvants. Suitable adjuvants include, for example, aluminium adjuvants, such as aluminium hydroxide or aluminium phosphate, Freund's Adjuvant, BAY, DC-chol, pcpp, monophoshoryl lipid A, CpG, QS-21, cholera toxin and formyl methionyl peptide (see, e.g., Vaccine Design, the Subunit and Adjuvant Approach, 1995, M. F. Powell and M. J. Newman, eds., Plenum Press, N.Y.). A vaccine of the present disclosure may comprise one of more adjuvants. Exemplary adjuvants include aluminium compounds (alum), N-acetylmuramyl-L-alanyl-D-glutamine and other adjuvants known to those of ordinary skill in the art.

The vaccines may be in soluble or microparticular form or may be formulated, for example, in liposomes. Where the vaccine is to be administered parenterally, e.g. by intravenous, cutaneous, subcutaneous, or other injection, the vaccine is typically in the form of a pyrogen-free, parenterally acceptable aqueous or oily solution or suspension. The preparation of parenterally acceptable solutions with suitable pH, isotonicity, stability, and the like, is within the skill in the art. Suitable diluents include, for example, water, phosphate buffered saline (PBS) and isotonic sodium chloride solution. In addition, sterile fixed oils may be employed conventionally as a solvent or suspending medium. For this purpose, any bland fixed oil may be employed including synthetic mono- or diglycerides. In addition, fatty acids such as oleic acid may likewise be used in the preparation of injectable preparations.

The present disclosure will now be described with reference to the following specific examples, which should not be construed as in any way limiting the scope of the disclosure.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The Feasibility of Using Coxiella Burnetii a Virulent Nine Mile Phase II Viable Bacteria as a Live Attenuated Vaccine Against Q Fever

A. Results

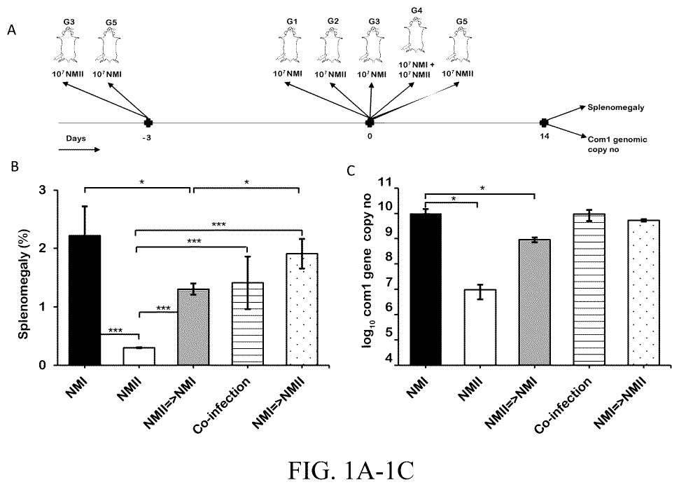

Viable avirulent NMII bacteria interfere virulent NMI infection. To explore whether viable avirulent NMII can interfere the infection caused by the virulent NMI, we examined i) if co-infection of mice with NMI and NMII would significantly affect the ability of NMI to cause disease and ii) if priming of mice with alive NMII before infection with NMI would alter the infection caused by the virulent NMI. Eight weeks old BALB/c mice were IP infected with 1)1×10⁷ virulent NMI, 2) 1×10⁷ avirulent NMII, 3) 1×10⁷ NMII 3 days before infection with 1×10⁷ NMI, 4) 1×10⁷ NMI+1×10⁷ NMII, or 5) 1×10⁷ NMI 3 days before infection with 1×10⁷ NMII. Splenomegaly and bacterial burden in the spleen were measured at 14 days post C. burnetii infection. As shown in FIG. 1B, compared to NMI-infected mice, splenomegaly was significantly reduced in NMI and NMII co-infected mice, and mice infected with avirulent NMII 3 days before infection with virulent NMI. In addition, splenomegaly in mice infected with NMI 3 days before infection with NMII was similar to NMI-infected mice. However, the bacterial burden was similar in the spleens among NMI-infected mice regardless infection with or without NMII bacteria (FIG. 1C). These results indicated that NMII bacteria interfered the severity of NMI infection induced disease but did not affect NMI bacterial replication in mice. Interestingly, the findings suggest that the viable non-pathogenic NMII bacteria may be capable to induce protective immunity against virulent C. burnetii infection.

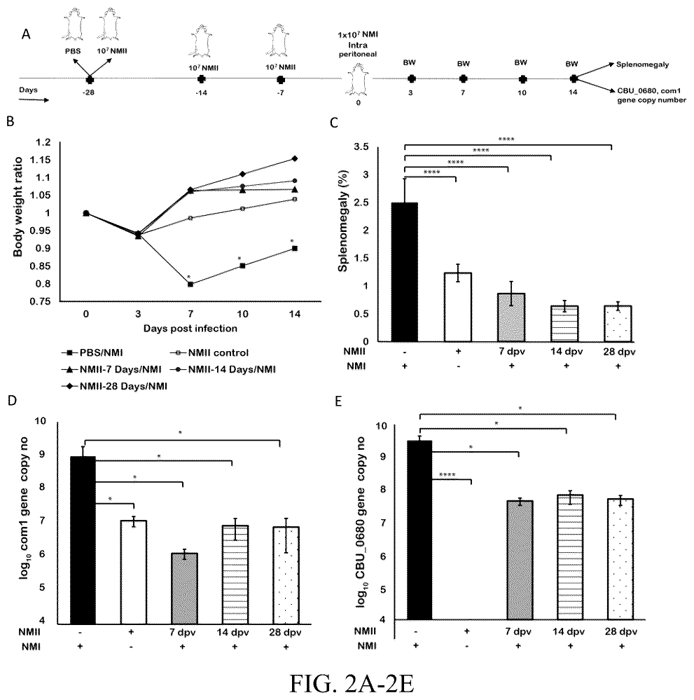

Mice immunization with viable NMII bacteria conferred significant protection against virulent NMI challenge. To confirm whether viable non-pathogenic NMII can induce protective immunity against virulent NMI infection, we examined if mice immunization with viable avirulent NMII bacteria would provide significant protection against virulent NMI challenge. Three groups of BALB/c mice were IP immunized with 1×10⁷ viable NMII bacteria and challenged with virulent NMI bacteria at 7, 14 and 28 days post-immunization with NMII, respectively. In addition, mice were infected with 1×10⁷ NMI or NMII bacteria and served as controls. Splenomegaly and bacterial burden in the spleen were measured at 14 days post virulent NMI challenge. As shown in FIG. 2B, compared to NMI-infected control mice, NMII-immunized mice protected NMI-infection induced body weight loss at all the time points post-immunization. In addition, splenomegaly and bacterial burden (com1 gene copy number) were significantly reduced in NMII-immunized mice at different time post-challenge with virulent NMI (FIGS. 2C and 2D). Further, to determine the NMI specific genomic copy number, CBU 0680 gene, which is not present in NMII, was quantified using RT-PCR. The results showed that CBU 0680 genomic copy number is similar to the coral genomic copy number (FIG. 2E), further supporting that virulent NMI bacterial burden in the spleens is significantly reduced in all NMII-immunized mice regardless the challenge time with virulent NMI. These results indicated that mice immunized with viable NMII bacteria were able to provide significant protection against virulent C. burnetii NMI challenge as early as 7 days post-immunization, suggesting that viable non-pathogenic NMII bacteria may be useful as a live attenuated vaccine against human Q fever.

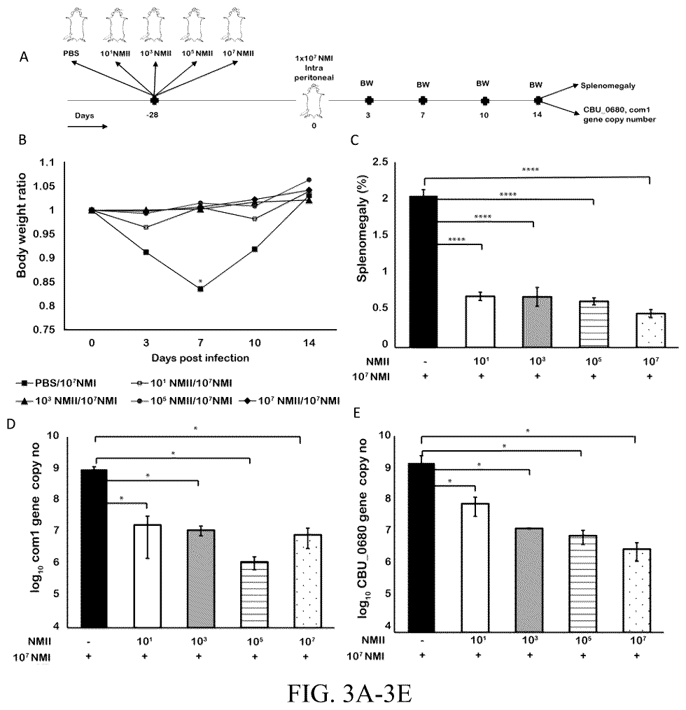

Mice immunized with lower doses of viable NMII bacteria also conferred significant protection against virulent C. burnetii challenge. To determine if immunization dose would affect the ability of non-pathogenic NMII bacteria to confer protection against virulent NMI infection, we tested whether mice immunization with different doses of viable NMI bacteria would affect their ability to confer protection against virulent NMI challenge. Eight weeks old BALB/c mice were IP immunized with 1) 1×10¹, 2) 1×10³, 3) 1×10⁵, or 4) 1×10⁷ of viable NMII bacteria and IP challenged with 1×10⁷ virulent NMI bacteria at 28 days post-immunization with avirulent NMII. In addition, mice were infected with 1×10⁷ NMI and served as unimmunized controls. Splenomegaly and bacterial burden in the spleen were measured at 14 days post virulent NMI challenge. As shown in FIG. 3B, compared to unimmunized control mice, all doses of NMII-immunized mice protected NMI-infection induced transitional body weight loss at 3, 7, and 10 days post NMI-infection. In addition, splenomegaly and bacterial burden were significantly reduced in NMII-immunized mice regardless the dose of immunization (FIGS. 3C, 3D, and 3E). The lowest dose of 1×10¹ viable NMI bacteria immunized mice also provided significant protection against virulent NMI challenge. These results further demonstrated that mice immunized with viable NMII bacteria could confer significant protection against virulent C. burnetii challenge.

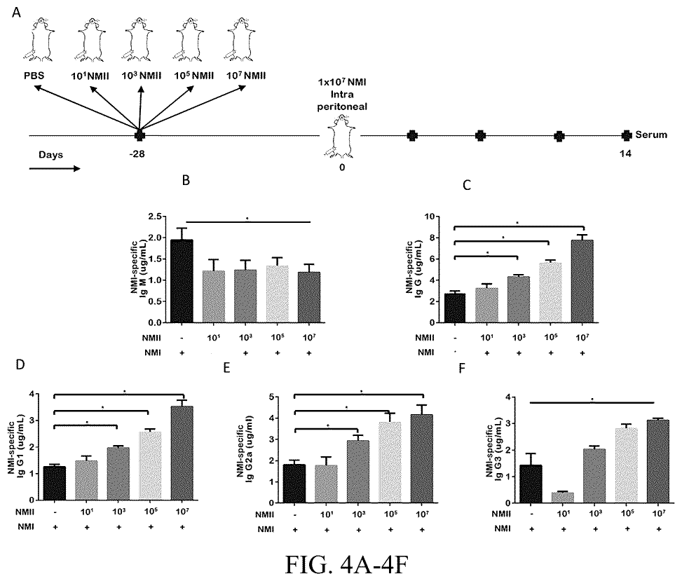

Viable NMII bacteria induced NMI-specific antibody response in mice. To determine if viable NMII bacteria would induce NMI-specific antibody response, immune sera were collected from mice immunized with 1) 1×10¹, 2) 1×10³, 3) 1×10⁵, or 4) 1×10⁷ of viable NMII bacteria at 14 days post-challenge with NMI in the above experiment for measuring the concentrations of anti-NMI specific IgM and IgG by ELISA. As shown in FIG. 4B, NMI-specific IgM titer in mice immunized with live NMII bacteria was significantly lower than unimmunized mice but there was no significant difference among different dose of NMII-immunized mice. In contrast, as shown in FIG. 4C, NMI-specific IgG titer was significantly higher in mice immunized with NMII except the lowest dose (10¹) of NMII and the pattern was similar for subclasses, IgG1 and IgG2a (FIGS. 4D and 4E). Interestingly, mice immunized with the lowest dose (10¹) of NMII exhibited lower level of IgG3 than all other groups including unimmunized group, however, there was no difference in protection, suggesting that IgG3 may not play a major role in NMII-mediated protection (FIG. 4F). Collectively, these results indicated that viable NMII bacteria induced a significant NMI-specific antibody response. Notably, although the lowest dose of 1×10¹ NMII-immunized mice induced a lower level of NMI-specific IgG response, they conferred a similar level of protection against NMI infection as other higher doses of NMII-immunized mice (FIGS. 3C and 3D), suggesting that NMI-specific IgG response may be not correlate to viable NMII bacteria induced protection.

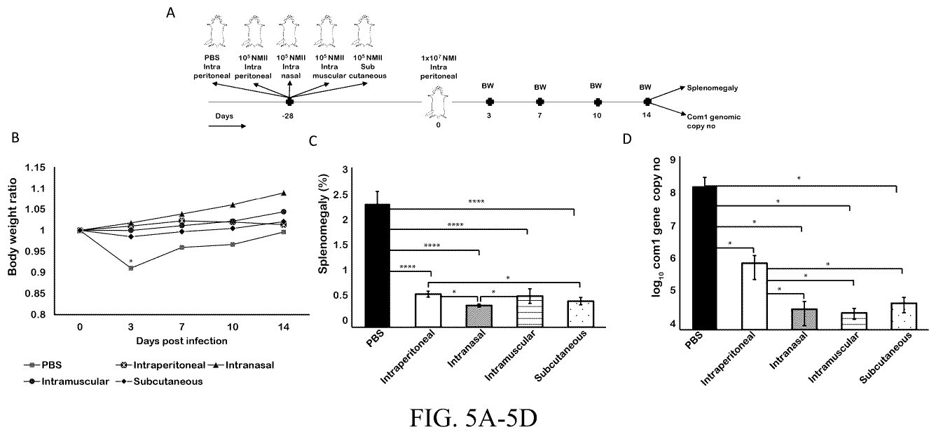

Mice immunization with viable NMII via different routes conferred similar protection against virulent NMI challenge. To identify the optimized route of immunization for viable NMII bacteria vaccine, we examined if route of immunization would affect the ability of viable NMII bacteria to confer protection against virulent NMI infection. BALB/c mice were IP, IN, IM or SQ immunized with 1×10⁵ of viable NMII bacteria. PBS vaccinated mice were maintained as unimmunized control. 28 days post vaccination, mice were IP challenged with 1×10⁷ of virulent NMI bacteria. Splenomegaly and bacterial burden in the spleen were measured at 14 days post NMI challenge. As shown in FIG. 5B, compared to unimmunized controls, all immunized mice protected the NMI-infection induced transitional body weight loss. Splenomegaly and bacterial burden were also significantly reduced in NMII-immunized mice regardless the route of immunization (FIGS. 5C and 5D). In addition, compared to IP immunized mice, splenomegaly and bacterial burden were significantly lower in IN and SQ immunized mice. Although there was no significant difference in splenomegaly between IP and IM immunized mice, bacterial burden in IM immunized mice was significantly lower than IP immunized mice. Collectively, the results indicated that IN immunization with viable NMII induced a stronger protective immunity against NMI infection than the other immunization routes. Since IN immunization route is less invasive than IP, IM and SQ immunization, it would be the optimized immunization route for human vaccination with live NMII vaccine.

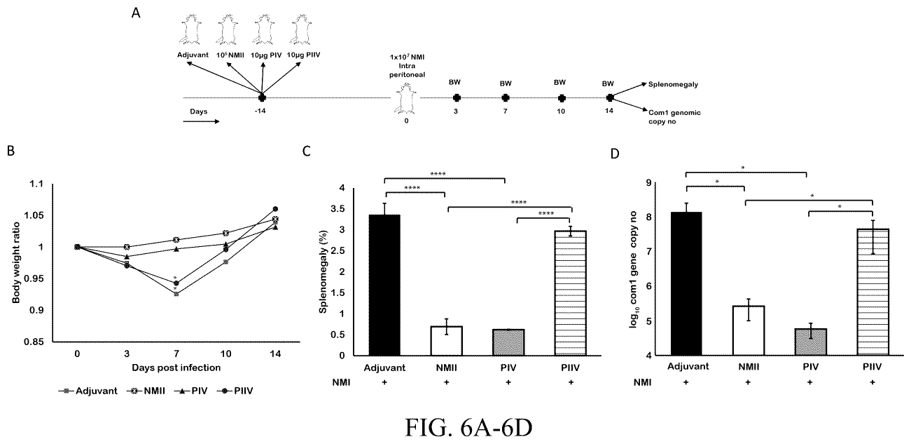

Viable NMII bacteria conferred a similar level of protection as the formalin-killed NMI vaccine (PIV). To determine if avirulent NMII live vaccine can confer a similar level of protection against virulent C. burnetii infection as the current licensed vaccine, Q-Vax, we compared the protective efficacy between viable NMII bacteria and PIV in BALB/c mice via SQ immunization. As shown in FIG. 6B, compared to adjuvant and formalin-killed NMII vaccine immunized mice, both viable NMII-immunized and PIV-immunized mice were able to protect NMI-infection induced transitional body weight-loss at 7 days post-challenge, and significantly reduced the splenomegaly (FIG. 6C) and bacterial burden in the spleens (FIG. 6D) at 14 days post-challenge at a comparable level. These results indicated that viable NMII bacteria induced a similar level of protection against virulent NMI infection as the PIV. In addition, the observation that viable NMII bacteria conferred significant protection against virulent NMI infection but killed-NMII vaccine did not confer a measurable protection, suggesting that bacterial replication in mice may be required for viable NMII-induced protective immunity.

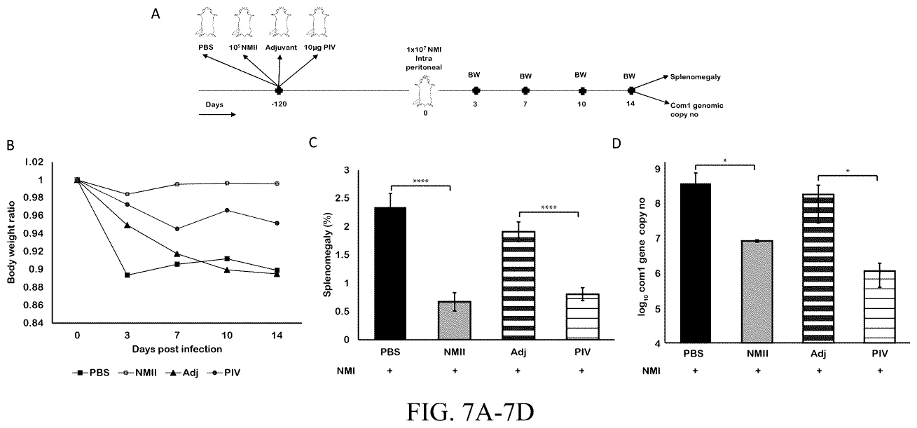

Viable NMII bacteria conferred a long-term protection against virulent NMI infection. According to our previous studies, 10 μg formalin-killed PIV provided highest levels of protection. To determine whether viable NMII bacteria can confer a similar level of long-term protection as the PIV, we compared the protective efficacy against virulent NMI infection between 10⁵ GE of viable NMII bacteria and 10 μg of PIV in BALB/c mice at 120 days post SQ immunization. As shown in FIG. 7, compared to unvaccinated and adjuvant immunized mice, both viable NMII-immunized and PIV-immunized mice were able to protect NMI-infection induced significant body weight-loss at different times post-challenge (FIG. 7B), and significantly reduced the splenomegaly (FIG. 7C) and bacterial burden in the spleens (FIG. 7D) at 14 days post-challenge at a comparable level. In addition, there was no significant difference in splenomegaly and bacterial burden in the spleens between viable NMII-immunized and PIV-immunized mice. These results demonstrated that viable NMII bacteria induced a similar level of long-term protection against virulent NMI challenge as the PIV. Notably, a nodule like inflammatory response was observed at the site of injection in PIV-immunized mice but the abnormal response at the site of injection did not appear in viable NMII-immunized mice. This observation indicates that immunization with viable NMII bacteria unlikely will induce adverse reactions as the PIV, suggesting that viable NMII bacteria might be useful as a live attenuated vaccine to replace the existing Q-Vax for prevention of human Q fever.

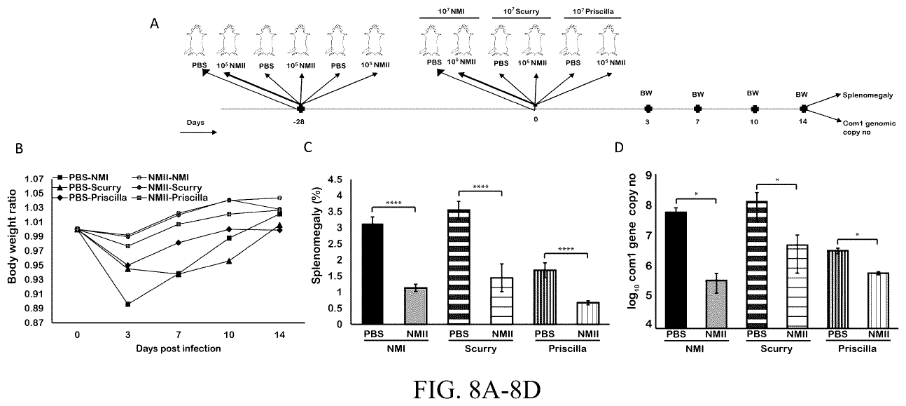

Viable NMII bacteria conferred a significant cross protection against various virulent C. burnetii isolates. The results from the above experiments demonstrate that viable NMII bacteria can elicit long-lasting significant protection against challenge with virulent NMI strain. However, it remains unknown if viable NMII bacteria can confer cross-protection against challenge with other virulent C. burnetii isolates. To address this question, we evaluated the cross-protective efficacy of viable NMII bacteria against virulent C. burnetii Priscilla and Scurry strains, which are chronic Q fever associated isolates. Three groups of BALB/c mice were IN immunized with 1×10⁵ of viable NMII bacteria and IP challenge with 1×10⁷ of virulent NMI, Priscilla or Scurry bacteria at 28 days post-immunization. In addition, three groups of PBS-immunized mice were infected with 1×10⁷ of NMI, Priscilla or Scurry bacteria and used as infection control for each respective strain. Splenomegaly and bacterial burden in the spleen were measured at 14 days after virulent C. burnetii infection. As shown in FIG. 8, compared to unimmunized and C. burnetii-infected control mice, viable NMII-immunized mice were able to protect NMI-, Priscilla- and Scurry-infection induced transitional body weight-loss at 3 days post-challenge (FIG. 8B), and significantly reduced the splenomegaly (FIG. 8C) and bacterial burden in the spleens (FIG. 8D) at 14 days post-challenge. These results demonstrated that IN immunization of mice with viable NMII bacteria conferred significant protection against various virulent C. burnetii isolate infection, suggesting that viable NMII bacteria can confer broad protection against challenge with heterogeneous isolates of virulent C. burnetii.

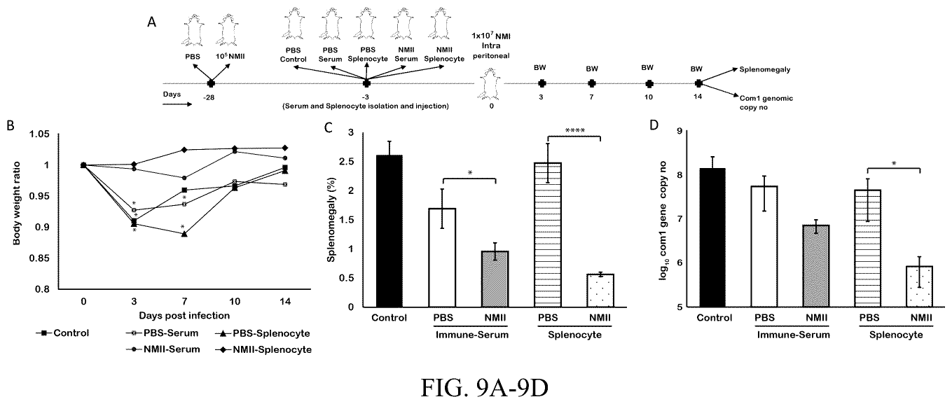

Immune sera and splenocytes from viable NMII-immunized mice provided a significant protection against NMI challenge in naive mice. To determine the roles of humoral and cellular immunity in viable NMII-induced protection, we tested if adoptive transfer of immune sera or splenocytes from NMII vaccinated mice can confer protection in naive mice against NMI infection. Immune sera and splenocytes were isolated from PBS or 1×10⁵ of viable NMII bacteria-immunized BALB/c mice at 14 days post-immunization and transferred to naive BALB/c mice by IP injection, respectively. Immune sera, splenocytes or PBS receiving mice were challenged with 1×10⁷ of NMI bacteria at 3 days after adoptive transfer. Splenomegaly and bacterial burden in the spleen were measured at 14 days post NMI challenge. Compared to mice received PBS, sera or splenocytes from PBS-immunized mice, mice receiving immune sera or splenocytes from NMII-immunized mice were able to protect NMI-infection induced significant body weight-loss at 3 and 7 days post-challenge (FIG. 9B) and significantly reduced the splenomegaly (FIG. 9C). In addition, compared to mice received PBS, sera or splenocytes from PBS-immunized mice, the bacterial burden in the spleens was significantly reduced in mice receiving splenocytes from NMII-immunized mice but was similar in mice receiving immune sera from NMII-immunized mice (FIG. 9D). These results suggest that both humoral and cellular immunities are involved in the viable NMII-induced protection but cellular immunity may play a critical role in controlling of bacterial replication in mice.

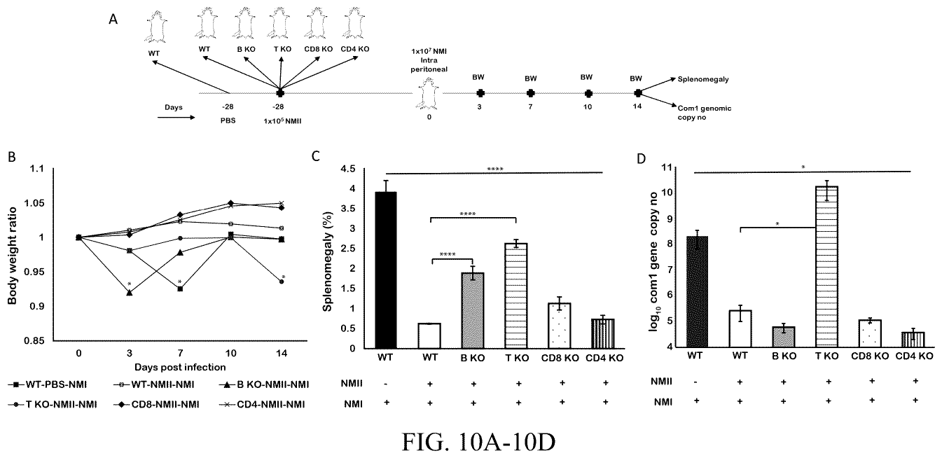

Viable NMII bacteria-induced protective immunity depends on both B cells and T cells but T cells may play a critical role in controlling bacterial replication. To further understand the mechanisms of viable NMII bacteria-induced protective immunity, we examined if B cell, T cell, CD4⁺ T cell or CD8⁺ T cell deficiency in mice will significantly affect the ability of viable NMII bacteria to confer protection against virulent NMI infection. C57BL/6J wild type (WT) and B cell, T cell, CD4⁺ T cell or CD8⁺ T cell deficient mice were IP immunized with viable NMII bacteria and challenged with NMI at 28 days post-immunization. Additionally, naive WT mice were infected with NMI and served as unimmunized controls. Splenomegaly and bacterial burden in the spleen were examined at 14 days post-challenge. As shown in FIG. 10B, NMI-infection induced a significant bodyweight loss in unimmunized WT, and NMII-immunized B cell deficient and T cell deficient mice at different times post-challenge but there was no bodyweight loss in NMII-immunized WT, CD4⁺ T cell deficient and CD8⁺ T cell deficient mice. In addition, compared to unimmunized WT mice, NMI-infection induced splenomegaly was significantly reduced in all NMII-immunized mice (FIG. 10C). However, although splenomegaly was similar between NMII-immunized B cell deficient and T cell deficient mice, it was significantly higher than NMII-immunized WT, CD4⁺ T cell deficient and CD8⁺ T cell deficient mice. As shown in FIG. 10D, the bacterial burden was comparable in the spleens from NMII-immunized WT, B cell deficient, CD4⁺ T cell deficient and CD8⁺ T cell deficient mice, but it was significantly higher in the spleen from NMII-immunized T cell deficient mice. Notably, the bacterial burden in the spleen from NMII-immunized T cell deficient mice even significantly higher than NMI-infected unimmunized WT mice. Collectively, these results demonstrated that viable NMII bacteria conferred a comparable level of protection against virulent C. burnetii challenge in WT, CD4⁺ T cell deficient and CD8⁺ T cell deficient mice, and a partial protection in B cell deficient and T cell deficient mice. The observation that the bacterial burden in the spleens from NMII-immunized T cell deficient mice was significantly higher than NMI-infected unimmunized WT mice suggests that T cells may play a critical role in controlling virulent C. burnetii replication in mice. Thus, Viable NMII bacteria-induced protective immunity depends on both B cells and T cells but T cells may play a critical role in controlling bacterial replication.

B. Material and Methods

Bacterial strains. C. burnetiid Nine Mile phase I (NMI) clone 7 (RSA 493), Nine Mile phase II (NMII) clone 4 (RSA 439), Scurry phase I (Q177) and Priscilla phase I (Q217) were propagated in acidified citrate cysteine medium-D (ACCM-D) as previously described [32]. Bacteria were purified by centrifugation at 15,000×g for 30 min, followed by two washes with sterile 1× phosphate-buffered saline (PBS) and stored at −80° C. until use. All phase I virulent strains used in this study undergo 4 passages and handled under biosafety level-3 conditions at the UTSA.

Bacterial quantification. Briefly, 200 μl of lysis buffer (1 M Tris, 0.5 M EDTA, 7 mg/ml glucose, 28 mg/ml lysozyme) and 10 μl of proteinase K (20 mg/ml) were added to 10 μl of bacterial stock and incubated at 60° C. for 18 h. Next, 21 μl of 10% SDS was added to samples and incubated at room temperature for 1 h. Finally, DNA was extracted using a High Pure PCR Template Preparation kit (Roche, Indianapolis, IN) as directed by the manufacturer. Bacterial load was determined using Taqman assay by quantifying C. burnetii com1 (FAM) (Custom Plus TaqMan™ RNA Assay, Invitrogen). The standard curve was generated using recombinant plasmid DNA (com1 gene ligated into pBluescript vector) and the results are represented genome equivalents (GE). Further, the bacterial viability was verified by serially diluting 100 of bacterial stock and plating in ACCM-D agarose plates and observing the colony forming units by C. burnetii after 7-10 days as previously described [32].

Formalin-killed bacteria and vaccine formulation. Purified C. burnetii NMI and NMII were inactivated for 48 h in 10% formalin, followed by dialysis in deionized water. Antigen concentrations were then measured using a BCA protein assay kit (Pierce, Rockford, IL) per the manufacturer's instructions and the concentration was represented as micrograms (μg). 10 μg of formalin-killed PIV and PIIV was dissolved in 504, PBS and mixed with 504, of alum adjuvant. The formulation was mixed well for 15 minutes.

Animal. Eight weeks old female BALB/c mice, B cell KO (Ighm^{tm1Cgn}) mice, T cell KO (FoxnI^{nu}) mice, CD4 KO (Cd4^{tmIMak}) and CD8 KO (Cd8a^{tmIMak}) mice were purchased from the Jackson Laboratory (Bar Harbor, ME). Since female mice are more resistant than males to C. burnetii infection, female mice were exclusively used in this study. All mice were housed in sterile microisolator cages containing four mice per cage under pathogen-free conditions at the UTSA animal biosafety level-3 laboratory facility. Animals were fed normally as per University regulations. All research protocols used in this study were approved by the institutional Biosafety committee and the Animal Care and Use committee of the UTSA (MU-CP001).

Immunization methods. Mice were immunized with viable NMII via intraperitoneal, intranasal, intramuscular or subcutaneous routes. Mice were anesthetized with isoflurane (5% induction, 1% to 2% maintenance) delivered in oxygen by using an anesthetic vaporizer before each immunization and challenge experiment. Since≥10⁷ of bacteria was used as optimized infectious dose for all virulent NMI infection in mice and to avoid unnecessary immune activation, we used 10⁵ GE of NMII as immunization dose in all immunization experiments if not specifically mentioned.

Intraperitoneal immunization (IP): 10¹, 10³, 10⁵ or 10⁷ GE of NMII bacteria were dissolved in 400 μL, PBS and injected using sterile 25G needle into the lower right quadrant of the animal's abdomen at 30° angle to a depth of 0.5 cm.

Intranasal immunization (IN): Using a sterile micropipette, 10⁵ GE of NMII bacteria were dissolved in 20 μL, PBS and slowly released into the nostrils.

Intramuscular immunization (IM): 10⁵ GE of NMII bacteria were dissolved in 20 μL, PBS and injected into the caudal thigh of the right pelvic leg to a depth of approximately 2 to 4 mm.

Subcutaneous immunization (SQ): Subcutaneous immunization was used to compare the protective efficacy of 10⁵ GE of viable NMII with 10 μg of formalin-killed PIV and PIIV. For subcutaneous injection, mice were restrained by scruff and the formulated vaccines were injected into the tent of skin over the scruff using a sterile 25G needle to a depth of 0.5 cm.

Serum and splenocyte isolation for adoptive transfer. Serum and splenocytes were collected from viable NMII-immunized mice and transferred to naive mice, respectively. Blood was collected through cardiac puncture from euthanized animal, incubated for 30 minutes at room temperature, centrifuged at 1500×g for 10 min at room temperature and the serum was collected. Splenocytes were isolated from BALB/c mice at 14 days post vaccination. Spleens were removed and homogenized. The cell suspension was then filtered through a 100-μm-pore-size nylon mesh to remove any connective tissue. Splenocytes were pelleted by centrifugation at 500×g for 8 min and suspended in 5 ml of ammonium chloride-potassium (ACK) lysis buffer for 5 min at room temperature to lyse red blood cells. Remaining cells were then pelleted by centrifugation at 500×g for 8 min and resuspended in 2 ml of fluorescence-activated cell sorting (FACS) buffer (PBS supplemented with 0.5% bovine serum albumin [BSA], 2 mM EDTA, and sodium azide) for counting. 1×10⁷ of splenocytes suspended in 100 μL of PBS was intraperitoneally (IP) injected into each mouse, while 500 μl of pooled serum was injected into each mouse. Three days after adoptive transfer, immune serum or splenocytes receiving and control mice were IP challenged with 1×10⁷ GE of NMI and observed for 14 days.

C. burnetii challenge and necropsy. For C. burnetii challenge, virulent NMI bacteria grown in ACCM-D were used. Mice were anesthetized and 10⁷ GE of NMI bacteria were dissolved in 400 μL PBS and was IP injected. Body weight was measured at 0, 3, 7, 10 and 14 days post-challenge. Mice were euthanized by CO₂ exposure at 14 days post NMI challenge. Spleen was dissected, weighed and 40 mg of spleen was used for genomic quantification. Blood was collected by cardiac puncture technique and serum was separated by spinning blood at 1500×g for 10 minutes at room temperature. If not processed immediately, spleen and serum were stored at −20° C. for further use.

qPCR. Spleen pieces were homogenized in 200 μl of lysis buffer (1 M Tris, 0.5 M EDTA, 7 mg/ml glucose, 28 mg/ml lysozyme) and filtered through a 100-μm-pore-size nylon mesh to remove any connective tissue. Ten microliters of proteinase K (20 mg/ml) was added to each sample prior to incubation at 60° C. for 18 h. Next, 21 μl of 10% SDS was added to samples and incubated at room temperature for 1 h. Finally, DNA was extracted using a High Pure PCR Template Preparation kit (Roche, Indianapolis, IN) as directed by the manufacturer. Bacterial burden was determined using Taqman assay by quantifying C. burnetii coral or CBU 0680 gene and normalized using mouse tfrc gene. Custom Plus TaqMan™ RNA Assay, FAM for coral or CBU 0680 was designed and procured from Invitrogen. TaqMan™ Copy Number Reference Assay, mouse, tfrc (VIC) was used to quantify the tfrc genes in mouse genome. The experiment was conducted using an Applied Biosystems QuantStudio3 real-time PCR system. The standard curve was generated using recombinant plasmid DNA (coral, CBU 0680, mouse tfrc genes ligated into pBluescript vector) and the results are represented as log₁₀ com1 gene copy number. All the experiments are performed at least in triplicates including both technical and biological replicates.

C. burnetii NMI-specific ELISA. Sera from vaccinated and unvaccinated control mice were used for quantification of total IgM, IgG, IgG1, IgG2a and IgG3 subclass antibodies. Microtiter plates (96-well) were coated with 100 μl of inactivated NMI antigen (0.5 μg/ml) or unlabeled anti-IgM or —IgG antibody (0.5 μg/ml, for the standard curve) (Southern Biotech, Birmingham, AL) in 0.05 M carbonate/bicarbonate coating buffer (pH 9.6) for 24 h at 4° C. Plates were blocked with 1% BSA in PBS-T buffer (0.05% Tween 20 in 1×PBS) and then incubated for 2 h with 200 μl of diluted sample serum (1:200 to 1:1,200) or serially diluted pure IgM, IgG, IgG1, IgG2a or IgG3 (Southern Biotech) at room temperature. Plates were washed four times with PBS-T buffer and then incubated with 100 μl of diluted horseradish peroxidase (HRP)-conjugated goat anti-mouse IgM, IgG, IgG1, or IgG2a (1:4,000 to 1:8,000) (Southern Biotech) at room temperature for 1 h. Plates were washed again four times with PBS-T, followed by the addition of 100 μl of 3,3′, 5,5′-tetramethylbenzidine (TMB) substrate (ThermoFisher Scientific). Reactions were stopped using 1 M H₃PO₄, and absorbance was measured at 450 nm using an Infinite F50 (Tecan, Switzerland) microplate reader.

Statistical analysis. Statistical analysis of body weight loss, splenomegaly and C. burnetii load in spleen was determined by Welch's unpaired t-test using GraphPad Prism 9.00 software (GraphPad). For all analysis, P<0.05 was deemed significant.

### REFERENCES

1. Centers for Disease C, Prevention DoH, Human S. Possession, use, and transfer of select agents and toxins; biennial review. Final rule. Fed Regist. 2012 2012/10//;77(194):61083-61115.
2. Angelakis et al. Int J Infect Dis. 2014 November;28:10⁷-10.
3. Eldin et al. Am J Trop Med Hyg. 2014 October;91(4):771-6.
4. Schimmer et al. Epidemiol Infect. 2012 January;140(1):27-35.
5. Morroy et al. PLOS ONE. 2015;10(7):e0131777.
6. Anderson et al. MMWR Recomm Rep. 2013 Mar. 29; 62(Rr-03):1-30.
7. Hatchette et al. Epidemiol Infect. 2003; 130(3):491-495.
8. Marmion et al. Epidemiol Infect. 1990; 104(2):275-287.
9. Baeten et al. PLOS ONE. 2018;13(10):e0205882.
10. Rekiki et al. Can J Vet Res. 2004; 68(3):226-228.
11. Arricau-Bouvery et al. Vaccine. 2005 Aug. 15; 23(35):4392-402.
12. Peng et al. J Immunol. 2012 Nov. 15; 189(10):4909-20.
13. Hackstadt, Ann N Y Acad Sci. 1990; 590:27-32.
14. Zhang et al. J Immunol. 2007 Dec. 15; 179(12):8372-80.
15. Beare et al. PLOS Pathogens. 2018;14(3):e1006922.
16. Millar et al. Genome Announc. 2017 Jun. 8; 5(23).
17. Beare et al. J Bacteriol. 2006 April; 188(7):2309-24.
18. Hoover et al. Infect Immun. 2002 December; 70(12):6726-33.
19. Vodkin and Williams, J Gen Microbiol. 1986 September;132(9):2587-94.
20 Hackstadt et al. Infect immun. 1985; 48(2):359-365.
21. Samuel and Hendrix, Curr Protoc Microbiol. 2009 November;Chapter 6:Unit 6C.1.
22. Vishwanath and Hackstadt, Infect immun. 1988; 56(1):40-44.
23. Cherla et al. Infect Immun. 2018 April;86(4).
24. Schoenlaub et al. Infect Immun. 2016 December; 84(12):3638-3654.
25. Long et al. Virulence. 2019 2019/01/01;10(1):133-150.
26. Moos and Hackstadt, Infect Immun. 1987 May; 55(5):1144-50.
27. Andoh et al., Infect Immun. 2007 July;75(7):3245-55.
28. van Schaik et al. Frontiers in cellular and infection microbiology. 2017; 7:25-25.
29. Shannon et al. PNAS. 2005;102(24):8722.
30. Vetter et al. Annals of Medicine. 2018 2018/02/17;50(2):110-120.
31. Bhardwaj S. Chapter 21—Vaccines. In: Vohora D, Singh G, editors. Pharmaceutical
32. Medicine and Translational Clinical Research. Boston: Academic Press; 2018. p. 341-353.
32. Sandoz et al. Appl Environ Microbiol. 2016; 82(10):3042-3051.
33. Ledbetter et al. Infect immun. 2020;88(3):e00824-19.
34. Scholzen et al. Front Immunol. 2019 2019-February-15;10(207).
35. Xiong et al. J Infect Dis. 2016; 215(10):1580-1589.
36. Gilkes et al. J Immunol. 2020;204(3):611.
37. Elliott et al. Infect Immun. 2013 December; 81(12):4604-14.
38. Dresler et al. Fron Microbiol. 2019 2019-September-18;10(2022).
39. Voth and Heinzen Cell Microbiol. 2007 April; 9(4):829-40.
40. Howe et al. Infect Immun. 2010; 78(8):3465.
41. Sellens et al. Vaccines (Basel). 2018;6(4):83.
42. Ranasinghe, Immunol Let. 2014 2014/10/01/;161(2):204-206.
43. Atkinson et al. Epidemiology and prevention of vaccine-preventable diseases. 12th ed. ed. [Atlanta, GA]: Dept. of Health and Human Services, Centers for Disease Control and Prevention; 2011. English.
44. Gilbert, Immunol. 2012; 135(1):19-26.
45. Izzo et al. J Infect Dis. 1988; 157(4):781-789.

1. A Q fever vaccine comprising a live avirulent Coxiella burnetii (C. burnetii) strain having a phase II LPS phenotype.

2. The vaccine of claim 1, wherein the avirulent C. burnetii strain is a nine mile phase II strain (NMII).

3. A method of immunizing a mammal against Q fever comprising administering a vaccine of claim 1.

4. The method of claim 3, wherein administering the vaccine is by intranasal administration.
